(19) United States
(12) Patent Application Publication
(10) Pub. No.: US 2004/0116742 A1
(43) Pub. Date: Jun. 17, 2004

(54) Selective reaction of hexafluoropropylene oxide with perfluoroacyl fluorides

(75) Inventor: Miguel Antonio Guerra, Woodbury, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 10/322,254

(22) Filed: Dec. 17, 2002

(51) Int. Cl. C07C 57/64; C 07C 3 9/82

(52) U.S. Cl. 562/825; 562/849

(57) ABSTRACT

A method is provided for reacting hexafluoropropylene oxide (HFPO) with a perfluoroacyl fluorides according to the formula X—R_{f}—COF (II) to selectively produce a monoaddition product according to the formula X—R_{f}—CF₂—O—CF(CF₃)COF (I), wherein selectivity for the monoaddition product over the biaddition product is 90% or greater or more typically 95% or greater. A continuous or repeated-batch process is provided comprising the steps of: a) providing a mixture of X—R_{f}—COF (II), a fluoride salt, and a polar solvent; b) adding hexafluoropropylene oxide (HFPO) in an amount such that X—R_{f}—COF remains in molar excess of HFPO by at least 10% and reacting X—R_{f}—COF with HFPO; c) separating unreacted X—R_{f}—COF from a mixture of addition products of hexafluoropropylene oxide (HFPO) and X—R_{f}—COF; and d) repeating step a) using unreacted X—R_{f}—COF separated in step c).

### FIELD OF THE INVENTION

This invention relates to a process for reacting hexafluoropropylene oxide (HFPO) with a perfluoroacyl fluorides according to they formula X—R_{f}—COF to selectively produce the monoaddition product according to the formula X—R_{f}—CF₂—O—CF(CF₃)COF with high utilization of reactants.

### BACKGROUND OF THE INVENTION

Reactions of hexafluoropropylene oxide (HFPO) with perfluoroacyl fluorides that result in a mixture of reaction products, differing in the number of incorporated HFPO units, are known. In the practice of these reactions, careful fractionation of the product mixture may be required to remove undesirable byproducts, such as products incorporating more than one HFPO unit.

U.S. Pat. No. 4,749,526 discloses preparations for fluoroaliphatic ether-containing carbonyl fluoride compounds by reacting a fluorinated carbonyl compound with hexafluoropropylene oxide in the presence of at least one catalyst selected from potassium iodide, potassium bromide, cesium iodide, cesium bromide, rubidium iodide and rubidium bromide.

### SUMMARY OF THE INVENTION

Briefly, the present invention provides a continuous or repeated-batch process for preparation of a compound according to formula (I): X—R_{f}—CF₂—O—CF(CF₃)COF, wherein X— is F—, FOC— or FSO₂— and wherein —R_{f}— is a linear, branched or cyclic fluoroalkene group containing 1-20 carbon atoms which is highly fluorinated and which may incorporate ether and tertiary amine groups, comprising the steps of: a) providing a mixture of X—R_{f}—COF (II), wherein X— and —R_{f}— are as defined for formula (I), a fluoride salt, and a polar solvent; b) adding hexafluoropropylene oxide (HFPO) in an amount such that X—R_{f}—COF remains in molar excess of HFPO by at least 10% and reacting X—R_{f}—COF with HFPO; c) separating unreacted X—R_{f}—COF from a mixture of addition products of hexafluoropropylene oxide (HFPO) and X—R_{f}—COF; and d) repeating step a) using unreacted X—R_{f}—COF separated in step c).

In another aspect, the present invention provides a method of reacting hexafluoropropylene oxide (HFPO) with perfluoroacyl fluorides according to the formula X—R_{f}—COF (II), wherein X and —R_{f}— are as described above, to form a mixture of addition products comprising the monoaddition product according to the formula X—R_{f}—CF₂—O—CF(CF₃)COF (I), wherein the molar amount of the monoaddition product is 90% or greater of the combined molar amount of the monoaddition product and a biaddition product according to the formula X—R_{f}—CF₂—O—CF(CF₃)CF₂—O—CF(CF₃)COF (III) in the mixture of addition products. More typically, the molar amount of the monoaddition product is 95% or greater of the combined molar amount of the monoaddition and biaddition products in the mixture of addition products.

What has not been described in the art, and is provided by the present invention, is an industrially useful method to react hexafluoropropylene oxide (HFPO) with perfluoroacyl fluorides which provides high selectivity for the monoaddition product, and, when excess perfluoroacyl fluoride is recycled, provides utilization of both HFPO and the perfluoroacyl fluoride reactant that approaches the level of selectivity, i.e., in excess of 90% and more typically in excess of 95%.

In this application, “highly fluorinated” means containing fluorine in an amount of 40 wt % or more, typically 50 wt % or more and more typically 60 wt % or more.

It is an advantage of the present invention to provide an industrially advantageous method of reacting hexafluoropropylene oxide (HFPO) with perfluoroacyl fluorides that provides utilization of both HFPO and the perfluoroacyl fluoride reactant that approaches 90% or higher.

### DETAILED DESCRIPTION OF THE PREFERRED EMBODIMENTS

The present invention provides a continuous or repeated-batch process for preparation of a compound according to formula (I): X—R_{f}—CF₂—O—CF(CF₃)COF, wherein X— is F—, FOC— or FSO₂— and wherein —R_{f}— is a linear, branched or cyclic fluoroalkene group containing 1-20 carbon atoms which is highly fluorinated and which may incorporate ether and tertiary amine groups, comprising the steps of: a) providing a mixture of X—R_{f}—COF (II), wherein X— and —R_{f}— are as defined for formula (I), a fluoride salt, and a polar solvent; b) adding hexafluoropropylene oxide (HFPO) in an amount such that X—R_{f}—COF remains in molar excess of HFPO by at least 10% and reacting X—R_{f}—COF with HFPO; c) separating unreacted X—R_{f}—COF from a mixture of addition products of hexafluoropropylene oxide (HFPO) and X—R_{f}—COF; and d) repeating step a) using unreacted X—R_{f}—COF separated in step c). In the reaction according to the present invention, the mixture of addition products comprises the monoaddition product according to the formula X—R_{f}—CF₂—O—CF(CF₃)COF (I), resulting from 1:1 combination of HFPO and X—R_{f}—COF, a biaddition product according to the formula X—R_{f}—CF₂—O—CF(CF₃)CF₂—O—CF(CF₃)COF (III), resulting from 2:1 combination of HFPO and X—R_{f}—COF, and potentially products resulting from 3:1, 4:1 and higher degrees of addition. Typically, the reaction according to the present invention is selective for the 1:1 product. Typically the molar amount of the monoaddition product is 90% or greater of the combined molar amount of the monoaddition (1:1) product and the biaddition (2:1) product, and more typically 95% or greater.

The perfluoroacyl fluoride reactant is a compound according to the formula:

X—R_{f}—COF (II)

wherein X— is F—, FOC— or FSO₂— and wherein —R_{f}— is a linear, branched or cyclic fluoroalkene group, typically a linear group, containing 1-20 carbon atoms, typically containing 1-10 carbon atoms, and more typically containing 2-4 carbon atoms, which is highly fluorinated, typically perfluorinated, and which may incorporate ether and tertiary amine groups, but typically incorporates no tertiary amine groups, more typically incorporates no ether or tertiary amine groups.

Any suitable reaction vessel may be used, as appropriate to a continuous or batchwise process. Typically, the process is a continuous or a repeating batch process, allowing for the recovery and reuse of perfluoroacyl fluoride reactant in subsequent repetitions of the reaction. The perfluoroacyl fluoride reactant is mixed with a fluoride salt in a polar solvent to form a pre-reaction mixture. Any suitable fluoride salt may be used, including salts of mono- or polyvalent cations and salts of polyatomic cations or, more typically, monoatomic cations, most typically KF. Typically the salt is provided in an amount of 0.1-10% by weight relative to the amount of perfluoroacyl fluoride reactant, more typically 1-5%, and most typically 2-4%. Any suitable polar solvent may be used. Typically the solvent is provided in an amount of 10-200% by weight relative to the amount of perfluoroacyl fluoride reactant, more typically 20-40%, and most typically 20-30%.

Hexafluoropropylene oxide (HFPO) is added to form a reaction mixture. HFPO is added in an amount such that X—R_{f}—COF remains in molar excess of HFPO by at least about 10%, more typically by at least about 20%, and most typically by at least about 30%. Typically, X—R_{f}—COF is in molar excess of HFPO by no more than 50% after addition of all HFPO.

The reaction mixture may be maintained at any suitable temperature and pressure. Typically, the reaction mixture is maintained at a temperature between −25° C. and 40° C., more typically between −25° C. and 25° C., and most typically between −20° C. and 0° C. Typically, the reaction mixture is maintained at a pressure between vacuum and 300 kPa, more typically between 20 and 110 kPa. HFPO may be added at any rate, provided that the temperature does not rise to a level that produces significant unwanted HFPO oligimerization. HFPO may be added very quickly if appropriate cooling apparatus are used.

After completion of the reaction, the unreacted X—R_{f}—COF is typically separated from the mixture of addition products by any suitable means, including solvent separation and distillation. Typically the unreacted X—R_{f}—COF thus recovered is used in a subsequent reaction.

The addition product mixture comprises the monoaddition product according to the formula X—R_{f}—CF₂—O—CF(CF₃)COF (I), resulting from 1:1 combination of HFPO and X—R_{f}—COF, a biaddition product according to the formula X—R_{f}—CF₂—O—CF(CF₃)CF₂—O—CF(CF₃)COF (III), resulting from 2:1 combination of HFPO and X—R_{f}—COF, and potentially, but not typically, products resulting from 3:1, 4:1 and higher degrees of addition. The product mixture may also include low levels, typically <1%, of HFPO dimer, trimer and higher oligomers. The reaction according to the present invention is selective for the 1:1 product, such that the molar amount of the monoaddition product is typically 90% or greater of the combined molar amount of the monoaddition (1:1) product and the biaddition (2:1) product, and more typically 95% or greater. HPFO, a valuable reactant, is productively consumed in an amount approaching but less than the reaction selectivity, since polyaddition of HPFO consumes a disproportionate amount of HPFO. Furthermore, when excess perfluoroacyl fluoride is recycled, utilization of perfluoroacyl fluoride reactant also approaches the reaction selectivity. The potential for reusing unreacted perfluoroacyl fluoride reactant, the high utilization of both reactants, approaching 90% or higher, and the elimination or reduction of cleanup of the monoaddition product all render the process according to the present invention highly useful in industrial applications.

This invention is useful in the industrial synthesis of HFPO-perfluoroacyl fluoride adducts.

Objects and advantages of this invention are further illustrated by the following examples, but the particular materials and amounts thereof recited in these examples, as well as other conditions and details, should not be construed to unduly limit this invention.

### EXAMPLES

Unless otherwise noted, all reagents were obtained or are available from Aldrich Chemical Co., Milwaukee, Wis., or may be synthesized by known methods.

### Example 1

113g KF and 1960 g of diglyme were charged in a 2-gallon (7571 ml) stainless steel reactor and stirred and cooled to −17° C. 3240 g (11.7 moles) of 4-(fluorosulfonyl)hexafluorobutyryl fluoride, FSO₂CF₂CF₂CF₂COF (M.W. 280.1), prepared by electrochemical fluorination of 1,4-Butane sultone as described in U.S. Pat. No. 2,732,398, was vacuum charged into the reactor and stirred for 30 minutes. (3240 g weight was net of 5807 g of 55.8% purity material.) 1485 g (8.95 moles) hexafluoropropylene oxide (HFPO) (M.W. 166.0), was added over one hour with the reaction temperature rising to −4 oC and pressure up to 28 kPa. The molar ratio was 11.7/8.95=1.29. The reaction mixture was stirred for 30 minutes and allowed to warm to room temperature. 7350 g of a bottom fluorochemical phase was recovered, containing 10% by weight unreacted acid fluoride, 45% by weight of the 1:1 addition product, perfluoro-4-(fluorosulfonyl)butoxypropionyl fluoride, FSO₂—CF₂CF₂CF₂CF₂—O—CF(CF₃)COF and 1.8% by weight of the 2:1 addition byproduct, FSO₂—CF₂CF₂CF₂CF₂—O—CF(CF₃)CF₂—O—CF(CF₃)COF. This result demonstrates an 83% yield based on HFPO (moles 1:1 addition product/moles HFPO reactant) and a desired selectivity for the 1:1 addition product of 96% (moles 1:1 addition product/moles 1:1 and 2:1 product).

### Example 1C (Comparative)

9 g KF and 200 ml of diglyme were charged in a 600 ml stainless steel reactor and stirred and cooled to 0° C. 180 g (0.64 moles) of 4-(fluorosulfonyl)hexafluorobutyryl fluoride, FSO₂CF₂CF₂CF₂COF (M.W. 280.1) was vacuum charged into the reactor and stirred for 30 minutes. (180 g weight was net of 400 g of 45% purity material.) 107 g (0.64 moles) hexafluoropropylene oxide (HFPO) (M.W. 166.0), was added over two and one half hours. The molar ratio was 0.64/0.64=1.00. The reaction mixture was stirred for 30 minutes and the bottom fluorochemical phase was distilled to give a 210 g fraction with a boiling point greater than 110° C. containing 75% by weight of the 1:1 addition product, 23% by weight of the 2:1 addition byproduct and 2% by weight of the 3:1 addition byproduct. This result demonstrates an 55% yield based on HFPO but a selectivity for the 1:1 addition product of 77%, comparing 1:1 and 2:1 addition products.

### Example 2

73 g KF and 3386 g of diglyme were charged in a 2-gallon (7571 ml) stainless steel reactor and cooled to −5° C. 1693 g (7.30 moles) of perfluoromethoxypropionyl fluoride, CF₃—O—CF₂CF₂COF (M.W. 232.0), prepared as described in U.S. Pat. No. 6,482,979, was vacuum charged into the reactor and stirred for 30 minutes. 848 g (5.11 moles) hexafluoropropylene oxide (HFPO) (M.W. 166.0), was added over fifteen minutes and allowed to react for an additional 15 minutes. The molar ratio was 7.30/5.11=1.43. The lower fluorochemical phase was distilled to give 1099 g of precut containing 26.2% starting acid fluoride and 16% of the desired 1:1 addition product, perfluoromethoxypropoxylpropionyl fluoride, CF₃—O—CF₂CF₂CF₂—O—CF(CF₃)COF. The product cut of 1533 g contained 90% by weight of the desired 1:1 addition product, and the final cut of 141 g was 66% 2:1 byproduct. This result demonstrates an 77% yield based on HFPO and a desired selectivity for the 1:1 addition product of 94%.

### Example 2C (Comparative)

4.3 g KF and 188 g of diglyme were charged in a 600 ml stainless steel reactor along with 112 g (0.48 moles) of perfluoromethoxypropionyl fluoride, CF₃—O—CF₂CF₂COF (M.W. 232.0) and the mixture was cooled to 0° C. 80.2 g (0.48 moles) hexafluoropropylene oxide (HFPO) (M.W. 166.0), was added over one hour at 10° C. The molar ratio was 0.48/0.48=1.00. After phase split, 182 g of fluorochemical was collected containing 78% of the desired 1:1 addition product and 23% of the 2:1 byproduct for an 74% yield based on HFPO and a selectivity of only 78%.

### Example 3

22 g KF and 300 g of diglyme were charged in a 2 liter 3-neck round bottom flask along with 1000 g (3.40 moles) of perfluoroadipoyl fluoride, FOC—CF₂CF₂CF₂CF₂—COF (M.W. 294.0), prepared by electrochemical fluorination of dimethyl adipate as described in U.S. Pat. No. 6,482,979, and the mixture was stirred for 30 minutes and cooled to −17° C. 480 g (2.89 moles) hexafluoropropylene oxide (HFPO) (M.W. 166.0), was added over one hour at 5° C. and the reaction mixture was stirred for an additional 30 minutes. The molar ratio was 3.40/2.89=1.18. 1490 g of a bottom fluorochemical phase was recovered, containing 69% by weight of the desired 1:1 addition product, perfluoro-6-(fluoroacyl)hexoxypropionyl fluoride, FOC—CF₂CF₂CF₂CF₂CF₂—O—CF(CF₃)COF and 8.8% by weight of the 2:1 addition byproduct, FOC—CF₂CF₂CF₂CF₂CF₂—O—CF(CF₃)CF₂—O—CF(CF₃)COF. This result demonstrates an 78% yield based on HFPO and a desired selectivity for the 1:1 addition product of 89%.

### Example 3C (Comparative)

33 g KF and 2140 g of diglyme were charged in a 5 liter 3-neck round bottom flask along with 1043 g (3.55 moles) of perfluoroadipoyl fluoride, FOC—CF₂CF₂CF₂CF₂—COF (M.W. 294.0) and the mixture was stirred for 30 minutes and cooled to 0° C. (1043 g weight was net of 1257 g of 83% purity material.) 587 g (3.54 moles) hexafluoropropylene oxide (HFPO) (M.W. 166.0), was added over three hour at room temperature. The molar ratio was 3.55/3.54=1.00. The mixture was distilled to give 1470 g of a mixture comprising 74% by weight of the desired 1:1 addition product and 26% by weight of the 2:1 addition byproduct. This result demonstrates an 67% yield based on HFPO but a desired selectivity for the 1:1 addition product of only 74%.

The reaction conditions for Examples 1, 1C, 2, 2C, 3, and 3C are summarized in Table I and the results are summarized in Table II. “NM” indicates “not measured.”

TABLE I

| | | reactant | reactant | reactant | HFPO | HFPO | HFPO | molar |
|---|---|---|---|---|---|---|---|---|
| Ex. | reactant | m.w. | wt (g) | moles | m.w. | wt (g) | moles | ratio |
| 1 | FSO₂— | 280.1 | 3,240 | 11.57 | 166.0 | 1485 | 8.95 | 1.29 |
| | C₃F₆—OCF | | | | | | | |
| 1C | FSO₂— | 280.1 | 180 | 0.64 | 166.0 | 107 | 0.64 | 1.00 |
| | C₃F₆—OCF | | | | | | | |
| 2 | CF₃O— | 232.0 | 1,693 | 7.30 | 166.0 | 848 | 5.11 | 1.43 |
| | C₂F₄—COF | | | | | | | |
| 2C | CF₃O— | 232.0 | 112 | 0.48 | 166.0 | 80.2 | 0.48 | 1.00 |
| | C₂F₄—COF | | | | | | | |
| 3 | FOC— | 294.0 | 1,000 | 3.40 | 166.0 | 480 | 2.89 | 1.18 |
| | C₄F₈—COF | | | | | | | |
| 3C | FOC— | 294.0 | 1,043 | 3.55 | 166.0 | 587 | 3.54 | 1.00 |
| | C₄F₈—COF | | | | | | | |

TABLE II

| | | 1:1 | 1:1 | 1:1 | | | 2:1 | 2:1 | 2:1 | Molar | reactant | reactant |
|---|---|---|---|---|---|---|---|---|---|---|---|---|
| | | product | product | product | Molar | | product | product | product | Selec- | collected | collected |
| Ex. | 1:1 product | m.w. | wt (g) | moles | Yield | 2:1 product | m.w. | wt (g) | moles | tivity | (g) | (moles) |
| 1 | FSO₂—C₄F₈—O— | 446.1 | 3,308 | 7.42 | 83% | FSO₂—C₄F₈—O— | 612.1 | 132 | 0.22 | 96% | 735 | 2.62 |
| | CF(CF₃)COF | | | | | CF(CF₃)CF₂—O— | | | | | | |
| | | | | | | CF(CF₃)COF | | | | | | |
| 1C | FSO₂—C₄F₈—O— | 446.1 | 239 | 0.54 | 55% | FSO₂—C₄F₈—O— | 612.1 | 73 | 0.12 | 77% | NM | NM |
| | CF(CF₃)COF | | | | | CF(CF₃)CF₂—O— | | | | | | |
| | | | | | | CF(CF₃)COF | | | | | | |
| 2 | CF₃O—C₃F₆—O— | 398.1 | 1,556 | 3.91 | 77% | CF₃O—C₃F₆—O— | 564.1 | 93 | 0.16 | 94% | 288 | 1.24 |
| | CF(CF₃)COF | | | | | CF(CF₃)CF₂—O— | | | | | | |
| | | | | | | CF(CF₃)COF | | | | | | |
| 2C | CF₃O—C₃F₆—O— | 398.1 | 141 | 0.35 | 74% | CF₃O—C₃F₆—O— | 564.1 | 41 | 0.07 | 78% | NM | NM |
| | CF(CF₃)COF | | | | | CF(CF₃)CF₂—O— | | | | | | |
| | | | | | | CF(CF₃)COF | | | | | | |
| 3 | FOC-C₅F₁₀—O— | 460.1 | 1,033 | 2.25 | 78% | FOC—C₅F₁₀—O— | 626.1 | 131 | 0.21 | 89% | NM | NM |
| | CF(CF₃)COF | | | | | CF(CF₃)CF₂—O— | | | | | | |
| | | | | | | CF(CF₃)COF | | | | | | |
| 3C | FOC-C₅F₁₀—O— | 460.1 | 1,088 | 2.36 | 67% | FOC—C₅F₁₀—O— | 626.1 | 382 | 0.61 | 74% | NM | NM |
| | CF(CF₃)COF | | | | | CF(CF₃)CF₂—O— | | | | | | |
| | | | | | | CF(CF₃)COF | | | | | | |

It can be readily seen that the process according to the present invention provides greatly improved selectivity for the 1:1 addition (monoaddition) product, often at greater yield. Furthermore, valuable unreacted perfluoroacyl fluoride reactant can be recovered for reuse, which renders the process according to the present invention highly useful in industrial applications such as continuous or repeated batch processes. When excess perfluoroacyl fluoride is recycled, utilization of perfluoroacyl fluoride reactant approaches the degree of reaction selectivity. Since HFPO, also a valuable reactant, is productively consumed in an amount slightly less than the reaction selectivity, utilization of both HFPO and the perfluoroacyl fluoride reactant approaches the level of selectivity, i.e., in excess of 90% and more typically in excess of 95%.

Various modifications and alterations of this invention will become apparent to those skilled in the art without departing from the scope and principles of this invention, and it should be understood that this invention is not to be unduly limited to the illustrative embodiments set forth hereinabove. All publications and patents are herein incorporated by reference to the same extent as if each individual publication or patent was specifically and individually indicated to be incorporated by reference.

We claim:

1. A continuous or repeated-batch process for preparation of a compound according to formula (I):
X—R_{f}—CF₂—O—CF(CF₃)COF (I)
wherein X— is F—, FOC— or FSO₂— and wherein —R_{f}— is a linear, branched or cyclic fluoroalkene group containing 1-20 carbon atoms which is highly fluorinated and which may incorporate ether and tertiary amine groups, comprising the steps of:
a) providing a mixture of:
X—R_{f}—COF (II)
wherein X— and —R_{f}— are as defined for formula (I), a fluoride salt, and a polar solvent;
b) adding hexafluoropropylene oxide (HFPO) in an amount such that X—R_{f}—COF remains in molar excess of HFPO by at least 10% and reacting X—R_{f}—COF with HFPO;
c) separating unreacted X—R_{f}—COF from a mixture of addition products of hexafluoropropylene oxide (HFPO) and X—R_{f}—COF;
d) repeating step a) using unreacted X—R_{f}—COF separated in step c).

2. The method according to claim 1 wherein said mixture of addition products comprise a monoaddition product according to formula (I):
X—R_{f}—CF₂—O—CF(CF₃)COF (I)
and a biaddition product according to formula (III):
X—R_{f}—CF₂—O—CF(CF₃)CF₂—O—CF(CF₃)COF (III)
wherein the molar amount of monoaddition product in said mixture of addition products is 90% or greater of the combined molar amount of said monoaddition product and said biaddition product.

3. The method according to claim 1 wherein said mixture of addition products comprise a monoaddition product according to formula (I):
X—R_{f}—CF₂—O—CF(CF₃)COF (I)
and a biaddition product according to formula (III):
X—R_{f}—CF₂—O—CF(CF₃)CF₂—O—CF(CF₃)COF (III)
wherein the molar amount of monoaddition product in said addition products is 95% or greater of the combined molar amount of said monoaddition product and said biaddition product.

4. The method according to claim 2 wherein molar yield of said monoaddition product relative to moles of HFPO added in step b) is 75% or greater.

5. The method according to claim 3 wherein molar yield of said monoaddition product relative to moles of HFPO added in step b) is 75% or greater.

6. The method according to claim 1 wherein step b) is carried out without addition of any catalyst other than said fluoride salt.

7. The method according to claim 1 wherein said fluoride salt is KF.

8. The method according to claim 6 wherein said fluoride salt is KF.

9. The method according to claim 1 wherein —R_{f}— is a linear, branched or cyclic perfluorinated fluoroalkene group containing 1-20 carbon atoms which may incorporate ether and tertiary amine groups.

10. The method according to claim 1 wherein —R_{f}— is a linear, branched or cyclic perfluorinated fluoroalkene group containing 1-20 carbon atoms.

11. A method of reacting hexafluoropropylene oxide (HFPO) with a perfluoroacyl fluorides according to the formula:
X—R_{f}—COF (II)
wherein X is F—, FOC— or FSO₂— and wherein —R_{f}— is a linear, branched or cyclic fluoroalkene group containing 1-20 carbon atoms which is highly fluorinated and which may incorporate ether and tertiary amine groups,
to form a mixture of addition products comprising the monoaddition product according to the formula:
X—R_{f}—CF₂—O—CF(CF₃)COF (I)
wherein the molar amount of said monoaddition product is 90% or greater of the combined molar amount of said monoaddition product and a biaddition product according to the formula:
X—R_{f}—CF₂—O—CF(CF₃)CF₂—O—CF(CF₃)COF (III)
in said mixture of addition products.

12. The method according to claim 11 wherein the molar amount of said monoaddition product is 90% or greater of the combined molar amount of said monoaddition product and said biaddition product in said mixture of addition products.

13. The method according to claim 11 wherein molar yield of said monoaddition product relative to moles of HFPO added in step b) is 75% or greater.

14. The method according to claim 12 wherein molar yield of said monoaddition product relative to moles of HFPO added in step b) is 75% or greater.

15. The method according to claim 11 wherein step b) is carried out without addition of any catalyst other than said fluoride salt.

16. The method according to claim 11 wherein said fluoride salt is KF.

17. The method according to claim 15 wherein said fluoride salt is KF.

18. The method according to claim 11 wherein —R_{f}— is a linear, branched or cyclic perfluorinated fluoroalkene group containing 1-20 carbon atoms which may incorporate ether and tertiary amine groups.

19. The method according to claim 11 wherein —R_{f}— is a linear, branched or cyclic perfluorinated fluoroalkene group containing 1-20 carbon atoms.
